(19) United States
(12) Patent Application Publication
(10) Pub. No.: US 2010/0152812 A1
(43) Pub. Date: Jun. 17, 2010

(54) SYSTEMS AND METHODS FOR PROMOTING NERVE RECOGNITION

(75) Inventors: J. Christopher Flaherty, Topsfield, MA (US); Jonathan T. Hartmann, Southborough, MA (US); Jessica L. Duda, Boston, MA (US); Shawn D. Wery, Duxbury, MA (US)

Correspondence Address: Mark J. Pandiscio;PANDISCIO & PANDISCIO, P.C., 470 Totten Pond Road, Waltham, MA, 02451, US

(73) Assignee: CYBERKINETICS NEUROTECHNOLOGY SYSTEMS, Foxborough, MA (US)

(21) Appl. No.: 12/305,421

(22) PCT Filed: Jun. 27, 2007
(86) PCT No.: PCT/US2007/072128

§ 371 Date: Feb. 16, 2010

Related U.S. Application Data
(60) Provisional application No. 60/816,620, filed on Jun. 27, 2006.

(51) Int. Cl. A61N 1/04; A61K 35/12; A61P 25/00

(52) U.S. Cl. 607/50; 424/93.7

(57) ABSTRACT

Various exemplary systems and methods for promoting nerve regeneration are disclosed. In certain exemplary embodiments, a nerve regeneration system may include a lead configured to be placed in a body proximate a damaged nerve, a portion of the lead being configured to stimulate the damaged nerve. The system may also include a control module configured to monitor a signal indicative of the nerve's response to the stimulation and adjust a parameter of the stimulation in response to the monitored signal.

This application claims the benefit of priority under 35 U.S.C. §119(e) of U.S. Provisional Application No. 60/816,620, filed Jun. 27, 2006, which is incorporated by reference herein in its entirety.

### FIELD OF THE INVENTION

The present disclosure relates generally to systems and methods for causing nerve cells to regenerate and, more particularly, to systems and methods for promoting nerve regeneration in the central and peripheral nervous systems of mammals.

### DESCRIPTION OF RELATED ART

The central nervous system, including the brain, is the primary control system of a body, communicating with one or more parts of the body via a complicated system of interconnected nerves. Nerves are cable-like bundles of axons that carry electrical signals and impulses between one or more neurons and the central nervous system. Thus, nerves play a critical role in communicating sensory and stimulatory signals between various parts of the body (e.g., muscles, organs, glands, etc.) and the central nervous system.

Nerves may be damaged or severed either through trauma or disease. Damaged or severed nerves may inhibit the central nervous system's ability to receive sensory and stimulatory data from individual neurons, potentially limiting the nervous system's control over the body. For example, severe nerve damage may lead to paralysis, such as paraplegia or quadriplegia.

In the case of the peripheral nervous system (i.e., the portion of the nervous system outside of the brain and spinal cord), damaged or severed nerve cells may have some natural regeneration. The nerve fibers grow across the injured area and extend through to their end target (e.g., skin, muscle, etc.). If the injured area is larger than a few millimeters, however, the nerve cells may not regenerate on its own and, if left untreated, permanent sensory loss and paralysis may ensue.

In the peripheral nervous system, a common treatment to repair damaged nerves involves a surgical procedure to harvest a healthy nerve from another part of the patient's body and graft the harvested nerve to bridge the damaged section. Although surgery can successfully repair damaged nerve cells in many cases, these procedures may have several disadvantages. For instance, in most cases, several invasive surgical procedures are required to find suitable donor nerves. Further, damage to nerves at the donor site is quite common, potentially leading to weakening of donor nerves at the expense of the recipient nerves.

Some alternatives to surgical repair of damaged nerves have been developed. These systems typically involve surrounding damaged nerves in a sheath and administering therapeutic drugs or electromagnetic energy to the damaged nerve site. The administration of the therapeutic drugs and/or electromagnetic energy may facilitate nerve regeneration, while the sheath guides the nerve to grow in a desired direction.

Although these systems provide promising alternatives to nerve grafting procedures, they may have several disadvantages. For example, many conventional nerve regeneration systems have limited data processing capabilities. Also, they do not include integrated devices that can deliver therapeutic agents (e.g., drugs, electromagnetic energy, etc.) and monitor biological or chemical responses to the delivered therapeutic agents. Instead, regeneration and growth of damaged nerves may require subsequent exploratory operations, which may be time consuming, costly, and invasive for the patient.

Options for repairing nerves in the central nervous system are much more limited. Currently, the only widely available treatment is to administer therapeutic drugs to the damaged nerves. Drug treatment for spinal injuries has had very limited success. Some developing treatments involve the use of stem cells and the application of simple electric fields, but these treatments have rendered few determinative results thus far.

Thus, there is a need for an improved nerve regeneration system that may overcome one or more of the problems discussed above. In particular, there is a need for an improved nerve regeneration system that can efficiently optimize the treatment parameters, without requiring invasive exploratory techniques.

### SUMMARY

Therefore, various exemplary embodiments of the invention may provide a nerve regeneration system that may include an interactive diagnostic device configured to measure nerve growth, re-growth, and/or connections between severed or otherwise damaged nerve segments.

To attain the advantages and in accordance with the purpose of the invention, as embodied and broadly described herein, one exemplary aspect of the invention may provide a nerve regeneration system comprising a lead configured to be placed in a body proximate a damaged nerve, a portion of the lead being configured to stimulate the damaged nerve. The system may also comprise a control module configured to monitor a signal indicative of the nerve's response to the stimulation and adjust a parameter of the stimulation in response to the monitored signal.

According to one exemplary aspect, the stimulation comprises a therapeutic electric signal, and the parameter of the stimulation may comprise a parameter associated with the electric signal. For example, the parameter may comprise one or more of strength, direction, current, or voltage of the electric signal.

According to another exemplary aspect, the nerve regeneration system may comprise an electrode coupled to the lead and configured to deliver electric stimulation to the damaged nerve. The electrode may include a plurality of electrodes and the parameter may comprise one or more of a number, a sequence, or a combination of electrodes to be energized to deliver electric stimulation. The system may also comprise a conductor for connecting the electrode to the control module.

According to still another aspect, the control module may be enclosed in a substantially sealed housing with one or more leads extending from the housing. The control module may be configured to communicate with an external device.

According to yet another aspect, the control module may be surgically implanted within the body or, alternatively, may be partially implanted in the body such that at least a portion of the control module is accessible from outside the body.

According to still another aspect, the control module may comprise a fluid delivery device configured to provide a therapeutic fluid to the damaged nerve. For example, the control module comprises a fluid port for supplying fluid to the fluid delivery device.

According to yet another aspect, the system may comprise a first lead may configured to deliver an electrical signal, and a second lead configured to deliver the therapeutic fluid to the damaged nerve. The lead may comprise a tube in fluid communication with the fluid delivery device and configured to deliver the therapeutic fluid to the damaged nerve.

According to another aspect, the present disclosure is directed toward a nerve regeneration system that comprises a nerve regeneration module comprising at least one lead implanted in a body proximate a damaged nerve. The nerve regeneration module may be configured to administer a nerve regeneration treatment to the damaged nerve and detect a patient response to the nerve regeneration treatment. The system may also include an interrogator communicatively coupled to the nerve regeneration module and configured to modify a parameter of the nerve regeneration treatment based on the detected patient response.

According to still another aspect, the nerve regeneration module is fully implanted in the body of a patient while the interrogator is outside the body. The interrogator is configured to modify the parameter of the nerve regeneration treatment after implantation of the nerve regeneration module.

According to yet another aspect, the interrogator may be wirelessly coupled, via a wireless communication device, to the nerve regeneration module. According to one embodiment, the interrogator comprises a personal data assistant (PDA) or a wireless telephone.

According to still another aspect, one or more leads of the nerve regeneration system may comprise an expandable member configured to secure the lead proximate the damaged nerve. For example, the expandable member may embody an inflatable balloon. Alternatively, one or more leads may comprise a protective sheath surrounding the at least one lead, the protective sheath including a barb for securing the at least one lead within the body of the patient.

According to still another aspect, the nerve regeneration system comprises a power supply configured to generate an electromagnetic signal for stimulating the damaged nerve. Accordingly, the at least one lead may comprise one or more electrodes electrically coupled to the power supply, the one or more electrodes being configured to deliver the electromagnetic signal to the damaged nerve. According to one embodiment, the one or more of the electrodes are disposed along a length of the at least one lead.

In accordance with yet another aspect, the present disclosure is directed toward a method for regenerating a damaged nerve. The method may comprise the steps of providing a therapeutic stimulation to a damaged nerve, monitoring a signal indicative of the nerve's response to the stimulation, and adjusting a stimulation parameter in response to the monitored signal.

According to one aspect, the steps of providing, monitoring, and adjusting are performed by an integrated device. The step of providing the therapeutic stimulation comprises implanting a stimulation device within a body, and adjusting the stimulation parameter after implantation.

According to still another aspect, the stimulation device comprises a control module having a substantially sealed housing with a lead extending from the housing. The lead may be fixed in the body of a patient proximate to the damaged nerve.

According to yet another aspect, the method may include the step of providing the monitored signal to an external diagnostic device, wherein adjusting the stimulation parameter comprises automatically adjusting the stimulation parameter based on the monitored signal. Adjusting the stimulation parameter may include manually adjusting the stimulation parameter based on the monitored signal. Alternatively, adjusting the stimulation parameter may comprise comparing the monitored signal with a predetermined threshold value, displaying results of the comparison on a display of the external device, and receiving a user command for adjusting the stimulation parameter.

According to yet another aspect, the step of adjusting the stimulation parameter may comprise comparing the monitored signal with a predetermined threshold value and performing a predetermined adjustment routine if the monitored signal exceeds an acceptable deviation limit from the predetermined threshold value.

According to still another aspect, providing a therapeutic stimulation comprises delivering an electromagnetic signal to the damaged nerve. Accordingly, adjusting a stimulation parameter comprises adjusting at least one of: a power level, a frequency, a field strength, and a field direction associated with the electromagnetic signal.

According to yet another aspect, the step of monitoring a signal may comprise providing an electrical test signal to the damaged nerve, measuring the nerve's response to the test signal, and determining a current location of a portion of the damaged nerve based on the measured response to the test signal. According to one embodiment, a growth of the nerve may be calculated as the difference between the current location of the portion of the damaged nerve and a previous location of the portion of the damaged nerve.

In accordance with yet another aspect, the present disclosure is directed toward a closed-loop nerve regeneration system that includes at least one lead implanted in a body proximate a damaged nerve and a control module connected to the at least one lead. The control module may be configured to administer a nerve regeneration treatment to the damaged nerve through the lead, monitor the growth associated with the damaged nerve, and determine whether to adjust a nerve regeneration treatment parameter based on the comparison of the monitored growth with the predetermined value.

According to yet another aspect, the present disclosure is directed toward a device for administering a neurological test to a portion of a patient's body. The device may comprise a probe, a drive assembly configured to move the probe relative to a patient's body, and a controller configured control the operation of the drive assembly to bring at least a portion of the probe in contact with the patient's body and to provide physical stimulation to the portion of the patient's body in accordance with a predetermined test parameter.

According to one exemplary aspect, the probe may comprise a pin for pricking the skin of the patient. Alternatively, the probe may include a rotatable member configured to rub the skin of the patient. Further, the drive assembly may comprise a linear drive device for extending and retracting the pin from the skin of the patient.

According to another exemplary aspect, the controller may be communicatively coupled to an external diagnostic device. The controller may be configured to control the operation of the drive assembly in response to command signals from the external diagnostic device.

According to still another exemplary aspect, the predetermined test parameter includes at least one of: a force applied by the drive assembly, an amount of pressure applied by the probe to the patient's body, an amount of movement of the probe relative to the patient's body, a duration of operation of the drive assembly, and a range of motion associated with the drive assembly.

According to yet another aspect, the system may comprise a fixing member configured to fix the device relative to the patient's body. The fixing member may comprise a band configured to wrap around a portion of the patient's body.

According to yet another aspect, the present disclosure is directed toward a method for administering a neurological test to a patient's body. The method may comprise the step of establishing at least one test parameter for administering a neurological test to the skin of the patient. The method may also include the steps of stimulating a surface of the patient's skin according to the at least one test parameter and monitoring the patient's response to the stimulation.

The at least one test parameter may include at least one of: a force applied by the drive assembly, an amount of pressure applied by the probe to the patient's body, an amount of movement of the probe relative to the patient's body, a duration of operation of the drive assembly, and a range of motion associated with the drive assembly.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention.

FIG. 1A illustrates an exemplary nerve regeneration system consistent with the disclosed embodiments.

FIG. 1B provides a schematic diagram illustrating various functional elements of the nerve regeneration system of FIG. 1A.

FIG. 2A provides a side view of an exemplary nerve regenerator in accordance with certain disclosed embodiments.

FIG. 2B provides a side view of an exemplary nerve regenerator adapted for partial subcutaneous implantation consistent with certain disclosed embodiments.

FIGS. 3A and 3B illustrate exemplary diagnostic tools for use with a nerve regeneration system consistent with the disclosed embodiments.

FIG. 4 provides a flowchart depicting an exemplary nerve regeneration process in accordance with the disclosed embodiments.

FIG. 5 provides a flowchart depicting an exemplary diagnostic and treatment process associated with an exemplary disclosed nerve regeneration system.

FIG. 6 provides a flowchart depicting another exemplary diagnostic and treatment process in accordance with certain disclosed embodiments.

FIG. 7 provides a flowchart depicting yet another exemplary diagnostic and treatment process in accordance with certain disclosed embodiments.

### DETAILED DESCRIPTION

Reference will now be made in detail to exemplary embodiments consistent with the invention, examples of which are illustrated in the accompanying drawings. Wherever possible, the same reference numbers will be used throughout the drawings to refer to the same or like parts.

The embodiments described herein are directed toward systems and methods for reconnecting diseased, severed, or otherwise damaged nerves. More specifically, the present embodiments provide a system for causing severed or damaged nerve axons to grow and re-attach to other healthy nerves. Accordingly, the nerve regeneration treatments described herein are directed toward restoring signal transmission capabilities of central and peripheral nervous systems to restore motor control and sensory functions of damaged nerves in patients.

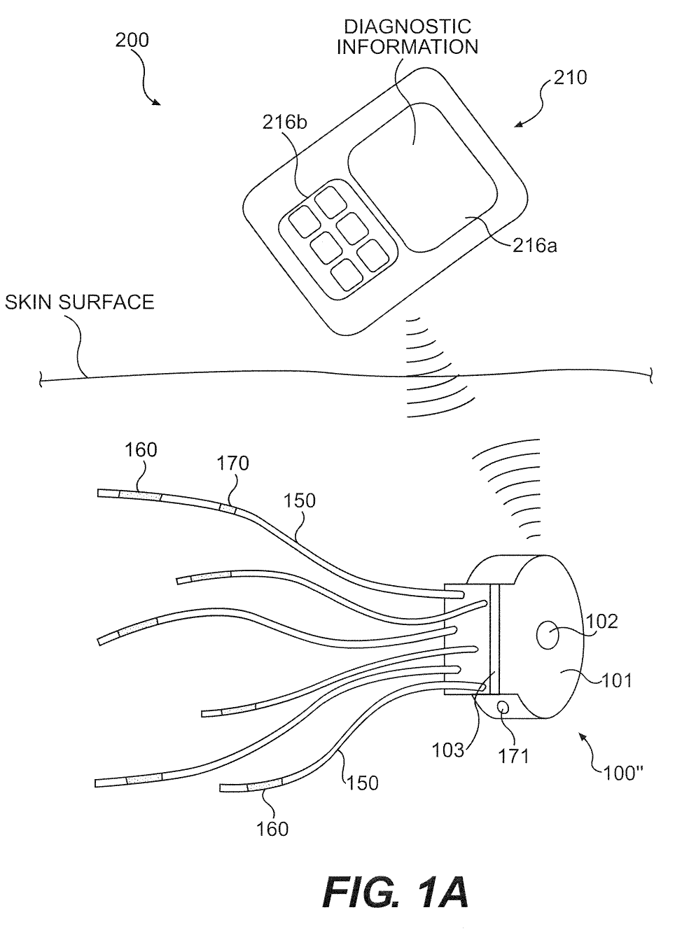

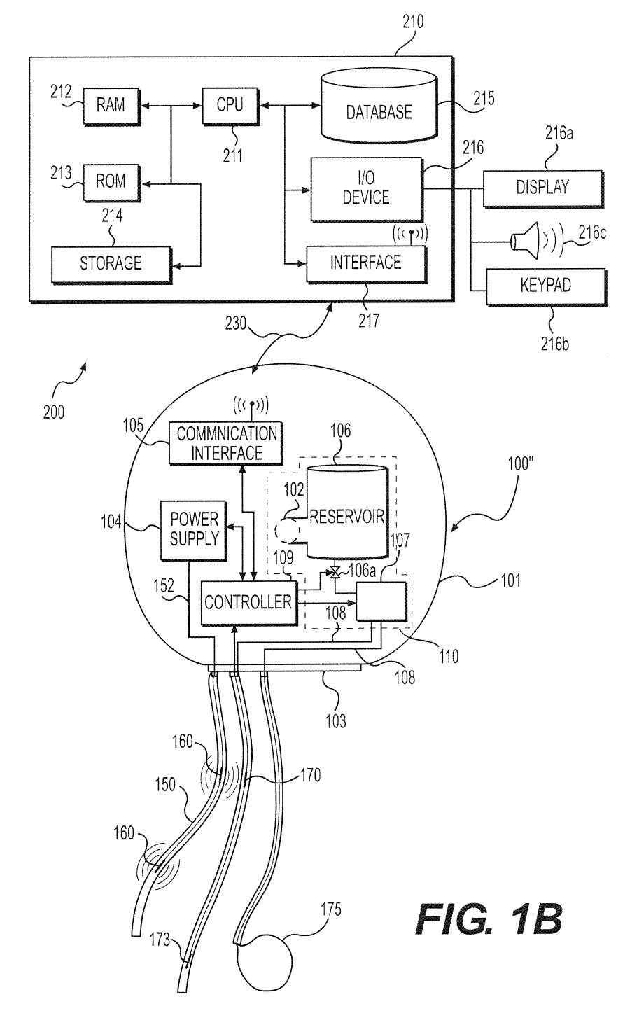

FIG. 1A illustrates an exemplary nerve regeneration system 200 consistent with the disclosed embodiments. Nerve regeneration system 200 may include one or more components that cooperate to regenerate nerves that have been diseased, damaged and/or severed. According to one embodiment, nerve regeneration system 200 may include a nerve regenerator 100″ for implantation in the body of patient at or near damaged nerve cells. Nerve regeneration system 200 may also include an interrogator 210 communicatively coupled to nerve regenerator 100″ and configured to communicate nerve treatment data with nerve regenerator 100″. Nerve treatment data may include, but not be limited to, control signals, diagnostic information, and other information associated with the administration of nerve regeneration treatments.

As illustrated in FIG. 1A, nerve regeneration system 200 may be configured to administer one or more nerve regeneration treatments, monitor nerve regeneration characteristics (e.g., biological, chemical, and/or electrical signals) in response to the administered treatment, and adjust one or more operational parameters of the nerve regeneration treatment based on the monitored characteristics. According to one embodiment, nerve regeneration system 200 may be configured to operate as an automated treatment and diagnostic system, whereby one or more parameters of nerve regeneration treatment are automatically adjusted, without requiring an external operator's intervention.

Alternatively or additionally, nerve regeneration system 200 may be operated in a “manual” mode. For example, nerve regenerator 100″ may be configured to administer a nerve regeneration treatment based on a control signal provided by a lab technician, doctor, nurse, or other authorized person via an external system (e.g., interrogator 210). During the administration of the treatment, nerve regenerator 100″ may collect patient data, such as nerve regeneration rate, nerve growth, data indicative of nerve response to various stimuli, etc. Nerve regenerator 100″ may provide these data to an external diagnostic system (e.g., interrogator 210) for analysis. Based on the analysis, a lab technician, doctor, nurse, or other authorized person may modify one or more treatment control parameters (e.g., stored in interrogator 210). Interrogator 210 then may subsequently transmit the updated control parameters to nerve regenerator 100″ via a wireless or direct data link. This diagnostic analysis and control cycle may continue during one or more treatment sessions until a desired nerve regeneration result is achieved.

Nerve regenerator 100″ may include a control module 101 that includes a plurality of electrical, mechanical, and/or electromechanical components for aiding in the administration, monitoring, and adaptation of one or more nerve regeneration therapies to damaged nerves. Control module 101 may include a fluid-tight housing having a fluid port 102 for receiving fluid (e.g., therapeutic drugs, air or other fluid for inflating lumens or other securing devices, etc.) for delivery to the patient's body. Control module 101 may also include one or more functional elements 171, such as transducers and/or sensors for monitoring one or more biological, chemical, and/or electrical conditions associated with the area surrounding control module 101. The number and type of components listed above are exemplary only and not intended to be limiting. For example, control module 101 may include additional, fewer, and/or different components than those listed above.

Nerve regenerator 100″ may include a plurality of leads 150 communicatively coupled to control module 101 via a header 103. Leads 150 may be flexible, tubular members that may be strategically placed at or near damaged nerves. Leads 150 may each include a hollow, flexible, insulating jacket constructed of plastic, rubber, silicone, or other flexible material. Leads 150 may provide a protective conduit for passing conductors and fluid delivery tubes to areas associated with damaged nerves. For example, leads 150 may provide a conduit for housing conductors that may be coupled to one or more electrodes 160 disposed along the length of leads 150. Alternatively and/or additionally, leads 150 may provide a conduit for housing fluid delivery tubes that may be coupled to one or more transducers 170 (e.g., a drug delivery element or an anchoring element) disposed along the length of one or more leads 150.

Leads 150 may be placed proximate damaged nerves. For example, leads 150 may be placed in and/or around the spinal cord of a patient with a spinal cord injury. Accordingly, the leads may be placed proximate damaged nerves of the central nervous system and may be situated such that a first electrode is on one side of a severed nerve and a second electrode is located on the other side. According to one embodiment, first and second electrodes may be placed equidistant from the damaged area (e.g., vertebral segments above and below spinal cord lesion).

As illustrated in FIG. 1A, nerve regenerator 100″ may be configured to be implanted within the body of a patient via a surgical procedure. Although nerve regenerator 100″ is illustrated as being completely implanted beneath the skin of a patient, it is contemplated that a portion of nerve regenerator 100″ may be located external to the body and/or at the surface of the skin. In one exemplary embodiment, control module 101 may be located at or near the surface of the skin, enabling easy access (e.g. via a syringe and needle) to fluid port 102 for delivering fluids to the control module 101. Regardless of whether nerve regenerator 100″ is implanted completely or partially within the body of the patient, leads 150 may be implanted and situated within the body of the patient at or near damaged nerves, thereby ensuring effective administration of nerve regeneration treatment to the damaged nerves.

Interrogator 210 may be communicatively coupled to nerve regenerator 100″ and configured to communicate information related to nerve regeneration treatment with nerve regenerator 100″. Interrogator 210 may also be configured to analyze treatment information, display treatment information to a patient, health care provider, and/or lab technician, and provide treatment recommendations based on the analyzed treatment information.

Interrogator 210 may include any type of diagnostic tool or computer system that may be adapted to communicate with nerve regenerator 100″. Interrogator 210 may include, for example, a handheld diagnostic tool, a personal desktop assistance (PDA), a wireless telephone or other communication device, a handheld computer gaming device, a desktop or notebook computer system, or any other processor-based device that is configured to execute diagnostic and/or control software associated with nerve regeneration system 200, receive data input from the user, and/or output data to the user via an interface. For example, as illustrated in FIG. 1A, interrogator 210 may embody a handheld communication device that includes a screen 216a for displaying diagnostic information to a user, a keypad 216b for receiving commands from the user, and one or more communication devices for wirelessly communicating data with nerve regenerator 100″. Although FIG. 1A illustrates interrogator 210 as being in wireless communication with nerve regenerator 100″, it is contemplated that interrogator 210 may communicate data to nerve regenerator 100″ via a wireline connection or direct data link (e.g., serial, parallel, USB, etc.). As such, interrogator 210 and nerve regenerator 100″ may each include data ports that support wire-based communication protocols.

According to an exemplary embodiment and as will be described in greater detail below, the presently disclosed nerve regeneration systems and associated methods involve passing electric current from at least one electrode to one or more other electrodes, providing a therapeutic electrical field therebetween. The field created between the electrodes may be an oscillating field generated by alternately applying positive and negative pulses of DC current between the electrodes. For example, a first electrode transmits DC current to a second electrode for a predetermined first time period to promote nerve growth in one direction. Subsequently, the polarity of the current is switched and the second electrode transmits the DC current to the first electrode to promote nerve growth in another direction. The DC current may be set at a predetermined level, such as between 200-1000 microamps (or other appropriate level). In an alternative embodiment, the DC current may vary during each pulse.

According to one embodiment, the duration of the pulses are established to be less than an axon “die back” period (i.e., the amount of time that an oppositely facing axon can withstand electric energy before beginning to degenerate). Die back periods have been estimated through experimentation to begin at time periods greater than one hour. According to another embodiment, the duration of the pulses are established to be at least 30 seconds such as to be long enough to cause axonal growth, as also has been estimated through experimentation.

In addition to reducing the die back in nerve axons, oscillating fields have been shown to reduce electrolysis and other toxin-producing nerve reactions that may be associated with electromagnetic fields. Furthermore, prolonged electric field exposure may, in some cases, adversely interfere with the effect of drugs and other types of nerve regenerative treatments. Accordingly, it may be advantageous to set pulse durations sufficiently long to promote nerve growth, while, at the same time, keeping the durations short enough to limit adverse effects associated with prolonged constant DC electric fields. According to one exemplary embodiment, pulse durations may initially be established at approximately thirty (30) seconds. This duration may be adjusted (e.g. increased) in accordance with the diagnostic methods, which are described in greater detail below.

FIG. 1B provides a schematic illustration of certain components and features associated with an exemplary nerve regeneration system 200 consistent with the disclosed embodiments. Specifically, FIG. 1B illustrates certain internal components associated with nerve regeneration system 200 and its constituent components and subsystems.

Control module 101 may include a housing that may be sealed to protect one or more components disposed inside the housing from the surrounding environment. Control module 101 may be made of a lightweight plastic, metallic (e.g., titanium), or composite material. According to one embodiment, control module 101 may be secured to a portion of the patient's body (e.g., skin, tissue, bone, etc.) using sutures, screws, or any other suitable device for fastening control module 101 to the patient's body. In embodiments where control module 101 is located outside of the patient's body, control module 101 may be secured onto the body using a strap or band.

Control module 101 may include a removable header 103 that provides an interface for passing electrical conductors or fluid delivery tubes through the wall of the housing of control module 101. Header 103 may be slidably coupled to a portion of the housing of control module 101. Alternatively, header 103 may be secured to the housing such as via screws or a welded joint.

Header 103 may include one or more interfaces for connecting leads 150. For example, header 103 may include a female, nut-type connector that may mate with a male, bolt-type connector associated with lead 150 to form a passage through header 103 for passing conductors and fluid delivery tubes therethrough. Header 103 may include any number of connection interfaces, providing access for several different leads. When not in use, the connection interfaces may be covered and/or sealed to protect control module 101 and any of its components from the surrounding environment.

In some exemplary embodiments, control module 101 may be configured to deliver electrical, magnetic, light energy and/or chemical stimulants to damaged nerve cells. For instance, as shown in FIG. 1B, control module 101 may include a power supply 104 configured to provide power to one or more components of control module 101; a communication interface 105 for transmitting patient data to and receiving control signals and configuration data from an external system (e.g., interrogator 210); a fluid delivery system that includes a reservoir 106 for storing fluid to be delivered to the patient's body and a fluid delivery device 107 for delivering fluid to the patient via one or more fluid delivery tubes 108; and a controller 109 for collecting, analyzing, controlling, monitoring, and/or storing information associated with the operation of control module 101.

Power supply 104 may include a battery, a fuel cell, a charge storing device, a transformer, a signal generator, an AC or DC power source, and/or any other device for providing power to operate control module 101. According to one embodiment, power supply 104 may include a rechargeable battery that may be inductively coupled to an external battery charger for charging the power supply. In some cases, power supply may be electrically coupled to an external power source via a power cable.

Power supply 104 may be communicatively coupled to one or more electrodes 160 via conductors 152. Electrodes 160 may embody high-conductivity metallic or metallic alloy materials such as platinum and/or platinum-iridium metals and may be adapted to deliver electrical energy to damaged nerves and/or tissue associated therewith. Electrodes 160 may be routed through lead 150 and, accordingly, may be strategically implanted at or near the damaged nerve sites.

Electrodes 160 may also include one or more micro-electrodes (not shown) protruding along the length of electrode 160. According to one embodiment, these micro-electrodes may include fibrous conductive materials (e.g., nanofibers, etc.) for enhancing the energy delivery capabilities associated with each electrode 160.

According to one embodiment, electrodes 160 may vary in length (e.g., from about 0.5 to 5 millimeters) and may have a relatively small diameter (e.g., a diameter of less than a human hair). As such, electrodes 160 may be small enough to be implanted in the spinal column and/or portions of the brain for delivering electro-therapeutic stimulants to portions of the central nervous system.

Communication interface 105 may include a communication module adapted to transfer information between control module 101 and an external diagnostic system, such as interrogator 210. Communication interface 105 may include an antenna to support wireless communication and/or a communication port to support direct connection to one or more external systems. In an exemplary embodiment, communication interface 105 may be adapted to support multiple wireless communication protocols such as, for example, Bluetooth, WLAN, cellular, other RF, and/or microwave communication formats. Alternatively or additionally, communication interface 105 may be adapted to support wire-based communication platforms and media such as, for example, serial (USB), parallel, Firewire, Ethernet, and optical communication platform or medium.

Fluid delivery system 110 may include one or more components for enabling fluid flow associated with nerve regeneration system 200. Fluid delivery system 110 may be configured to dispense therapeutic drugs (e.g., pain killers, nerve growth agent, proteins and fluids for promoting healthy nerve growth environment, etc.) to the patient's body. Fluid delivery system 110 may also be configured to deliver fluids for inflating one or more balloons adapted to secure leads 150 and/or control module 101 in a particular location.

As mentioned above, fluid delivery system 110 may include reservoir 106 in fluid communication with fluid port 102 and fluid delivery device 107 configured to deliver fluid stored in reservoir 106 to one or more transducers 170 via one or more fluid delivery tubes 108. Fluid port 102 may enable delivery of fluids to the control module 101, without requiring removal or disassembly of the control module 101. In some exemplary embodiments, fluid port 102 may include a re-sealable membrane, such as, for example, a silicone septum or other composite membrane, adapted to re-seal after a puncture by a hypodermic or other anti-coring needle. Although FIG. 1 is illustrated as having a single fluid port 102, additional fluid ports and/or drug delivery mechanisms may be provided. For example, if multiple therapeutic drugs are required as part of a nerve regeneration treatment, the fluid delivery system may include multiple fluid ports 102 and/or drug delivery mechanisms to allow separate injection and/or handling of the drugs in the system.

Reservoir 106 may be in fluid communication with fluid port 102 and configured to store the fluid delivered to fluid port 102. Reservoir 106 may embody a fluidly isolated compartment for storing a supply of fluids for use by fluid delivery system 110. Although control module 101 is illustrated as having a single reservoir, additional reservoirs 106 may be provided. For example, in an exemplary embodiment, the fluid delivery system may include at least a first reservoir and a second reservoir. The first reservoir may contain nerve growth agent, while a second reservoir may contain a photoreactive, luminescent and/or radiolabeled dye that, when injected into the body and exposed to a detecting device such as a phototransmiter and camera or a radiographic detector such as a fluoroscope, may aid in observing nerve activity and/or nerve regenerative growth during and/or after therapeutic treatments.

Fluid delivery device 107 may control the fluid flow associated with nerve regenerator 100″. According to one embodiment, fluid delivery device 107 may include a pump operatively coupled to controller 109 and adapted to operate in response to command signals received from controller 109. Fluid delivery device 107 may be coupled to reservoir 106 via a valve 106a, which may be operated by controller 109 to enable fluid flow from reservoir 106 to fluid delivery device 107. When multiple reservoirs 106 are used, a group of reservoirs may be selectively coupled to fluid delivery device 107 via a single controller-operated valve. Accordingly, by selectively coupling one or more reservoirs 106 to fluid delivery device 107 using valves (e.g., valve 106a) on an ad hoc basis, a single delivery device may be used to dispense multiple fluids required by nerve regeneration system 100″, reducing costs and implant size typically needed for multiple fluid delivery devices.

Fluid delivery device 107 may be fluidly coupled to one or more fluid delivery tubes 108, which may be routed through leads 150. When nerve regenerator 100″ is implanted, fluid delivery tubes 108 and/or leads 150 may be placed in desired locations proximate the damaged nerves. Fluid delivery tubes 108 may be terminated in one or more needles or other flow conduits that protrude from lead 150 for depositing fluid (e.g., therapeutic drugs) to damaged nerve sites. Alternatively or additionally, fluid delivery tubes 108 and/or leads 150 may include openings, or a porous material to release fluid into the damaged nerve sites. Alternatively or additionally, an electromagnetic field may be generated to deliver drugs or other agents via iontophoresis. Alternatively or additionally, fluid delivery tubes 108 may be used to deliver stem cells to the damaged nerve sites.

In addition to dispensing therapeutic drugs, fluid delivery system 110 may be used to secure nerve regenerator 100″ and/or one or more leads 150 in the desired location. For example, in an exemplary embodiment, fluid delivery system 110 may include one or more inflatable balloons 175 attached to the end of fluid delivery tube 108, which may be coupled to the fluid delivery device 107. When fluid is delivered to balloon 175, balloon 175 inflates, thereby securing leads 150 in place. These balloons may substantially prevent nerve regenerator 100″ and/or one or more leads 150 from excessive movement in the body.

As explained, the fluid delivery system 110 may include a separate reservoir 106 containing a filler agent (e.g., air, saline, etc.) and fluid delivery device 107 delivers the filler agent to inflatable balloons 175. Alternatively or additionally, fluid delivery device 107 may also be adapted to dispense materials that aid in determining the effectiveness of nerve regeneration treatments. For example, fluid delivery device 107 may dispense light sensitive fluids or dyes that, when exposed to light or suitable electromagnetic radiation (e.g., generated by an LED, optical, RF, or microwave generator associated with one or more leads 150), may aid in detecting nerve endings. Alternatively or additionally, fluid delivery device 107 may dispense a radiolabeled isotope or other radiographic material that, when imaged by a fluoroscope, may aid in visualizing nerves and/or nerve growth. By measuring nerve ending locations periodically, a growth rate of the nerve endings may be determined.

Controller 109 may include any type of microcontroller or processor-based device that may be configured to control one or more operational aspects of nerve regenerator 100″. According to one exemplary embodiment, controller 109 may be operated manually or automatically. For example, in a manual operating mode, controller 109 may be configured to receive commands from an external device (e.g., interrogator 210) for operating nerve regenerator 100″ via communication interface 105. Alternatively, in an automated mode, controller 109 may be configured to control the operations of nerve regenerator 100″ without requiring separate commands from the external device. In either case, controller 109 may be adapted to store and/or transmit operation data associated with nerve regenerator 100″, treatment data associated with a patient, and other information related to nerve regeneration treatments for later analysis by interrogator 210 or other suitable diagnostic device.

Controller 109 may be electrically coupled to power supply 104 and configured to regulate power output to components associated with nerve regenerator 100″. Additionally, controller 109 may include electronic switching and logic circuitry for operating power supply 104 to provide electromagnetic stimulation via electrodes 160 to damaged nerves. According to one embodiment, controller 109 may be adapted to control the voltage and/or current levels provided by power supply 104. In addition, controller 109 may be configured to control the frequency of the electromagnetic stimulation generated by power supply 104.

Controller 109 may also be configured to control an oscillating electromagnetic field (e.g. a switching DC field, such as a constant current DC field created by flowing approximately 200-1000 microamps from a first electrode, through tissue, to a second electrode) for stimulating nerve regeneration. As explained, controller 109 may be electrically coupled to power supply 104, which may include a signal generator for generating an electromagnetic field. According to one embodiment, controller 109 may be configured to control the frequency, period, and amplitude of the oscillating electromagnetic field so as to minimize degeneration of anodally facing axons and to stimulate growth of cathodally facing axons. Accordingly, the electromagnetic field generated by power supply 104 may be adjusted by controller 109 so as to maximize the growth rate of nerves facing a first direction, without desensitizing or damaging nerves facing a different direction (e.g. an opposite direction).

Controller 109 may also be electrically coupled to fluid delivery device 107 to control the delivery of fluids associated with nerve regenerator 100″. For example, controller 109 may be configured to provide control signals for operating reservoir selecting valves 106a. Alternatively or additionally, controller 109 may be configured to operate fluid delivery device 107 to deliver therapeutic drugs to damaged nerves and/or to inflate/deflate balloon 175.

Controller 109 may be in data communication with one or more sensors 173 and may be configured to receive/collect information associated with nerve treatment, including biological, physiological, chemical, and/or electrical data associated with the patient. Sensors 173 may include, for example, temperature sensors, voltage and/or current sensors (e.g., EKG sensors, EEG sensors, etc.), chemical sensors (e.g., glucose sensors, blood sensors, etc.), radiation sensors, magnetic sensors, or any other type of sensors adapted to collect data associated with a patient response to nerve regeneration treatment. Data received by sensors 173 may be collected in controller 109 and provided to interrogator 210 through communication interface 105 via communication link 230.

Communication link 230 may include any network or data link that provides two-way communication between nerve regenerator 100″ and an external diagnostic system, such as interrogator 210. For example, communication link 230 may communicatively couple nerve regenerator 100″ to interrogator 210 across a wireless networking platform such as, for example, a cellular, Bluetooth, microwave, point-to-point wireless, point-to-multipoint wireless, multipoint-to-multipoint wireless, or any other appropriate communication platform for networking a number of components. Although communication link 230 is illustrated as a wireless communication link, communication link 230 may include wireline links such as, for example, serial, parallel, USB, fiber optic, waveguide, or any other type of wired communication medium.

As explained, interrogator 210 may be a processor-based system on which processes and methods consistent with the disclosed embodiments may be implemented. For example, as illustrated in FIG. 1B, interrogator 210 may include one or more hardware and/or software components configured to execute computer programs. The computer programs may include, for example, diagnostic software for analyzing nerve regeneration treatments, evaluating the effectiveness of the treatments, modifying one or more parameters of the treatments, and/or controlling operation of nerve regenerator 100″.

For example, interrogator 210 may include one or more hardware components such as, for example, a central processing unit (CPU) 211, a random access memory (RAM) module 212, a read-only memory (ROM) module 213, a storage 214, a database 215, one or more input/output (I/O) devices 216, and an interface 217. Alternatively or additionally, interrogator 210 may include one or more software components such as, for example, a computer-readable medium including computer-executable instructions for performing methods consistent with certain disclosed embodiments. It is contemplated that one or more of the hardware components listed above may be implemented using software. For example, storage 214 may include a software partition associated with one or more other hardware components of interrogator 210. Interrogator 210 may include additional, fewer, and/or different components than those listed above. It is understood that the components listed above are exemplary only and not intended to be limiting.

CPU 211 may include one or more processors, each configured to execute instructions and process data to perform one or more functions associated with interrogator 210. As illustrated in FIG. 1B, CPU 211 may be communicatively coupled to RAM 212, ROM 213, storage 214, database 215, I/O devices 216, and interface 217. CPU 211 may be configured to execute sequences of computer program instructions to perform various processes, which will be described in detail below. The computer program instructions may be loaded into RAM for execution by CPU 211.

RAM 212 and ROM 213 may each include one or more devices for storing information associated with an operation of interrogator 210 and/or CPU 211. For example, ROM 213 may include a memory device configured to access and store information associated with interrogator 210, including information for identifying, initializing, and monitoring the operation of one or more components and subsystems of interrogator 210. RAM 212 may include a memory device for storing data associated with one or more operations of CPU 211. For example, ROM 213 may load instructions into RAM 212 for execution by CPU 211.

Storage 214 may include any type of mass storage device configured to store information necessary for CPU 211 to perform processes. For example, storage 214 may include one or more magnetic and/or optical disk devices, such as hard drives, CD-ROMs, DVD-ROMs, or any other type of mass media device.

Database 215 may include one or more software and/or hardware components that cooperate to store, organize, sort, filter, and/or arrange data used by interrogator 210 and/or CPU 211. For example, database 215 may include historical treatment settings (e.g., drug dosages, drug delivery schedules, electromagnetic treatment schedules, electromagnetic treatment power settings, etc.), nerve regeneration data (e.g., nerve growth rate, etc.), patient treatment response data (e.g., EKG data, EEG data, etc.), and/or any other type of data that may be used to diagnose and/or control nerve regenerator 100″. CPU 211 may access the information stored in database 215 for comparing the current treatment levels (and patient responses associated therewith) with historical treatment levels to establish a nerve regeneration treatment. Alternatively or additionally, historical data may be used to customize threshold levels used in the analysis of patient data. Thus, threshold levels for patients that experience greater nerve regeneration may be set higher than threshold levels for patients whose nerve regeneration rate lags behind a normal level, enabling more aggressive treatment options for highly responsive nerves. It is contemplated that database 215 may store additional and/or different information than that listed above.

I/O devices 216 may include one or more components configured to communicate information with a user associated with interrogator 210. For example, I/O devices may include a console with an integrated keypad 216b and/or mouse to allow a user to input parameters associated with interrogator 210. I/O devices 216 may also include a display 216a including a graphical user interface (GUI) for outputting information on a monitor. I/O devices 216 may also include peripheral devices such as, for example, a printer for printing information associated with interrogator 210, a user-accessible disk drive (e.g., a USB port, a floppy, CD-ROM, or DVD-ROM drive, etc.) to allow a user to input data stored on a portable media device, a microphone, a speaker system 216c, or any other suitable type of interface device.

Interface 217 may include one or more components configured to transmit and receive data via a communication network, such as the Internet, a local area network, a workstation peer-to-peer network, a direct link network, a wireless network, or any other suitable communication platform. For example, interface 217 may include one or more modulators, demodulators, multiplexers, demultiplexers, network communication devices, wireless devices, antennas, modems, and any other type of device configured to enable data communication via a communication network.

Interrogator 210 may be configured to provide an interface that allows users (e.g., patient, health care provider, etc.) to modify one or more nerve regeneration treatment parameters after implantation of nerve regenerator 100″ into the patient's body. Interrogator 210 may include software that provides users with an interface screen that includes one or more user-adjustable treatment parameters (e.g., drug dosage, drug delivery schedule, electromagnetic treatment schedule, electromagnetic field parameters (e.g., voltage level, electric and magnetic field direction, etc.)). Once established, users may upload the control parameters onto controller 109 associated with control module 101. Accordingly, controller 109 may administer the treatment in accordance with the user-defined parameters.

In some situations, nerve regenerator 100″ and/or one or more devices associated therewith may require periodic configuration and/or calibration to operate properly. Accordingly, interrogator 210 may also be configured to initiate a configuration and/or calibration subroutine for nerve regenerator 100″ and/or its constituent components. For example, should a sensor 173 for measuring electrical signals associated with nerve cells become out of calibration (e.g., as identified by an unrecognizable signal and/or excessive amount of electrical noise in the detected signal), interrogator 210 may be configured to calibrate the electrical sensor by providing a test signal and adjusting a sensor parameter (e.g., gain, etc.) associated with the sensor to cancel or filter any excessive noise. In addition, interrogator 210 may be configured to initiate a reset sequence for restoring one or more parameters associated with nerve regenerator 100″ to a default (e.g., factory/manufacturer preset) condition.

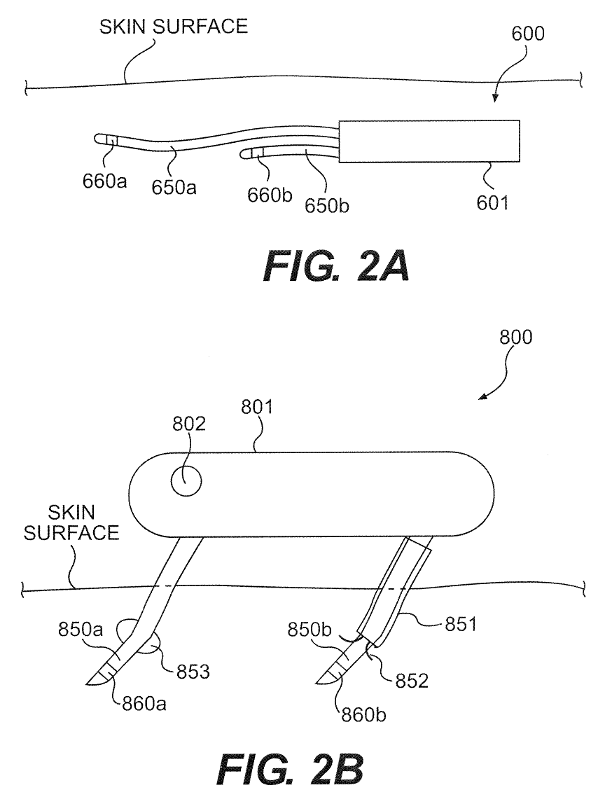

As explained, nerve regenerators may be fully or partially disposed in the body of a patient. FIGS. 2A and 2B illustrate exemplary configurations of nerve regenerators, consistent with the disclosed embodiments. FIG. 2A illustrates a nerve regenerator 600 that is fully implanted within the body of the patient, and FIG. 2B illustrates a nerve regenerator 800 that is partially implanted in the body of the patient.

FIG. 2A provides an exemplary side view of control module 601, leads 650a and 650b, and electrodes 660a and 660b. According to the embodiment of FIG. 2A, electrodes 660a and 660b may be adapted to provide electrical energy for stimulating nerves and surrounding tissue and, when not providing stimulating energy, may be configured to sense data associated with a patient response (e.g. a patient physiologic response) to the nerve regeneration treatment. For example, electrodes 660a and 660b may be configured to deliver electromagnetic pulse energy (e.g. a constant current DC field generated by current passing from electrode 660a to electrode 660b and the tissue in between) generated by a power supply or signal generator associated with control module 101. During periods between pulses and/or during energy delivery, electrodes 660a and/or 660b may be configured to detect biological, chemical, and/or electrical feedback in response to the pulses or other stimulation provided. Alternatively or additionally, nerve regenerator 600 may be arranged with separate sensing devices for detecting patient response data. According to this embodiment, electrodes 660a and 660b may be exclusively dedicated to electromagnetic stimulation treatment, while the sensors may be dedicated exclusively to the detection and monitoring of patient response data. Alternatively or additionally, control module 601 may provide a non-electromagnetic stimulation treatment such as drug or other agent delivery, or stem cell delivery.

As illustrated in FIG. 2A, leads 650a and 650b (and potentially the electrodes associated therewith) may have different lengths. This feature may be particularly advantageous for determining the nerve growth rate associated with damaged or severed nerves. For example, electrical stimulation may be provided to the damaged nerves by each of electrodes 660a and 660b (e.g. a constant current DC field generated by current passing from electrode 660a to electrode 660b and the tissue in between). The electrical stimulation may prompt an electrical or other response from the damaged nerves. The response may be measured by electrodes 660a and 660b and/or another sensor (not shown). Using signal analysis software, the response signals may be analyzed and mapped to determine the positions of the nerves. The determined positions may be analyzed to measure the growth of the measured nerves. Also, electrodes 660a and 660b may be calibrated in such a manner that changes in the strength of the responses received by each of the electrodes 660a and 660b may correspond to changes in the position of one or more ends of a severed nerve. By measuring these positional changes over time, a nerve growth rate may be determined.

FIG. 2B illustrates an exemplary side view of a partially implanted nerve regenerator 800, consistent with certain disclosed embodiments. As illustrated in FIG. 2B, partially implanted nerve regenerator 800 includes a control module 801 that may be located externally from the body of the patient, with leads 850a and 850b and/or electrodes 860a and 860b extending (percutaneously) through the skin and terminating in an area proximate damaged nerves. Because control module 801 is located outside the body of the patient, the embodiment illustrated in FIG. 2B is much less invasive than implanting the entire nerve regenerator beneath the skin.

However, by exposing a large portion of nerve regenerator 800 to various external forces, leads 850a and 850b may be susceptible to undesired movement and/or dislodgement, potentially resulting in reduced effectiveness and/or damage to electrodes 860a and 860b. To prevent the movement of leads 850a and 850b within or from the body of the patient, one or more mechanisms for securing leads 850a, 850b may be implemented. For example, according to one embodiment, nerve regenerator 800 may include an inflatable balloon 853. Upon installation of lead 850a in a desired location, balloon 853 may be inflated with filling material (e.g., air, saline, etc.) via fluid port 802, essentially anchoring lead 850a in place.

According to another embodiment, nerve regenerator 800 may include an outer sheath 851 covering one or more leads (such as lead 850b). The outer sheath may include one or more projection barbs 852. Projection barb 852 may be a resiliently biased anchor device made of, for example, Nitinol or spring metal and may be expanded or contracted to anchor lead 850b in a desired location. Alternatively or additionally, projection barb 852 may embody a retractable electrode or drug-delivery needle-type device for administering a nerve regeneration treatment. These devices may be selectively retracted using a mechanical transducer (e.g., screw-type projection device that is expanded/collapsed by rotating the outer sheath relative to the lead).

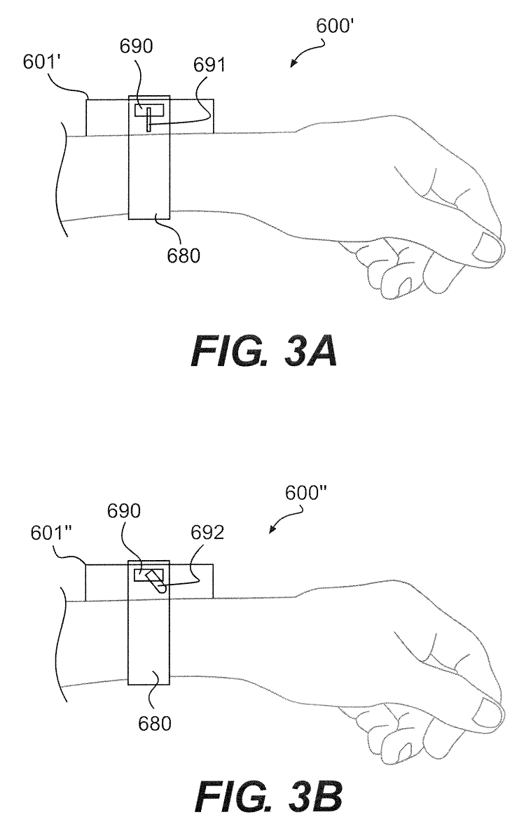

FIGS. 3A and 3B provide alternate embodiments of a diagnostic tool consistent with the disclosed embodiments. For example, FIG. 3A illustrates a diagnostic tool 600′ that administers a pin-prick test (e.g. a standard neurological pin-pick test or similar) to the skin of a patient to monitor a patient's response to certain sensory stimulants. FIG. 3B illustrates a diagnostic tool 600″ that administers a light touch test (e.g. a standard neurological light touch test or similar) to the skin of a patient to monitor a patient's neural response from a different set of nerves than that tested by diagnostic tool 600′ of FIG. 3A.

As illustrated in FIG. 3A, diagnostic tool 600′ includes a device configured to insert the tip of a pin 691 against the skin of a patient to perform a sensory recovery test specifically in a more reliable and repeatable manner than is currently available to clinicians and patients. Diagnostic tool 600′ may include a control module 601′ including a plurality of electrical components, such as circuit boards for controlling the administration of the pin-prick test, batteries for powering one or more devices associated with the tool, sensors for measuring the depth of pin into the patient's skin, and/or a linear motor for inserting and extracting the pin during the administration of the test.

Diagnostic tool 600′ may also include a band 680 for precisely holding diagnostic tool 600′ in place while administering the pin-prick test. Band 680 may include a flexible fabric that may be wrapped and secured to hold diagnostic tool 600′ against a patent's body (e.g., arm, leg, etc.) in an area of the body that includes damaged nerves. For example, band 680 may include flexible Velcro, elastic nylon, or any other type of flexible material that can be used to hold diagnostic tool 600′ in place during the administration of the test.

Diagnostic tool 600′ may also include a linear drive assembly 690 for inserting and extracting the pin while the diagnostic test is being performed. Linear drive assembly 690 may include a pin 691, coupled to a portion of the linear drive assembly 690. One or more of linear drive assembly 690 and/or pin 691 may include safety devices, which may be used to limit the depth that the pin 691 is inserted into the patient's skin. Safety devices may also embody electronic control devices that may be set to limit the time, frequency, and speed with which the test is administered. For example, diagnostic tool 600′ may include an optical sensor for measuring the distance between the pin 691 and the skin to determine when the pin 691 comes in contact with the skin of the patient and preventing the pin 691 from penetrating too deeply into the skin.

FIG. 3B illustrates a diagnostic tool 600″ for administering a light-touch test to a patient specifically in a more reliable and repeatable manner than is currently available to clinicians and patients. The light-touch test may be employed to test a patient's response to lighter (and potentially less intrusive) neural stimulants. For example, while the pin-prick diagnostic tool illustrated in FIG. 3A may be used to determine whether a patient has regained certain sensory capability and feeling, the light-touch test is used to determine whether a patient has regained different sensory nerve functions. As such, the stimulation provided by the light touch test of FIG. 3B may be used to modify treatment in a different manner than the stimulation of the pin-prick diagnostic tool of FIG. 3A.

Similar to diagnostic tool 600′ of FIG. 3A, diagnostic tool 600″ of FIG. 3B may include a control module 601″, band 680, and motor drive assembly 690. Diagnostic tool 600″ may include a rotatable probe 692 coupled to motor drive assembly 690 and configured to lightly contact the skin of a patient, reliably and repeatably mimicking the motion of a clinician's finger in a standard light touch test. Diagnostic tool 600″ may also include one or more sensors for determining parameters associated with the administration of the light touch test. For example, diagnostic tool may include an optical sensor for detecting the distance between rotatable probe 692 and the skin of the patient, a pressure sensor for determining the amount of force applied during the test, and/or any other type of sensor that may aid in the administration of the diagnostic test.

Although diagnostic tools 600′ and 600″ are illustrated as being used to administer a diagnostic test on the forearm of a patient, it is contemplated that diagnostic tools 600′ and 600″ may be used on any other part of the patient's body (e.g. a portion of the patient's body associated with a nerve regeneration treatment). Accordingly, components and/or component parameters may be modified to facilitate the administration of the test. For example, users may modify the force applied by rotatable probe 692 during performance of the light-touch test.

As explained, nerve regeneration system 200 may include one or more components for administering various therapeutic treatments to damaged nerves. Therapeutic treatments may include providing electromagnetic stimulation (e.g. a constant current DC field which changes polarity at a period of more than thirty (30) seconds and less than one (1) hour), administering therapeutic drugs, stem cells or other agents, and/or a combination of agent delivery and energy stimulation. The type and combination of treatment administered, the length of treatment, and the body's adaptive response to the treatment may each contribute to the effectiveness of a given treatment on the nerve regeneration. As such, nerve regeneration system 200 may be configured to monitor the patient's biological, physiological, chemical, and/or electrical responses to the administered nerve regeneration treatments. Nerve regeneration system 200 may also be configured to determine the effectiveness of the nerve regeneration treatment and modify at least one parameter of the nerve regeneration treatment to enhance the effectiveness of the nerve regeneration treatment. In addition, processes and features consistent with the disclosed embodiments may provide methods in which users can modify operational parameters of the nerve regeneration device after implantation into the body of the patient.

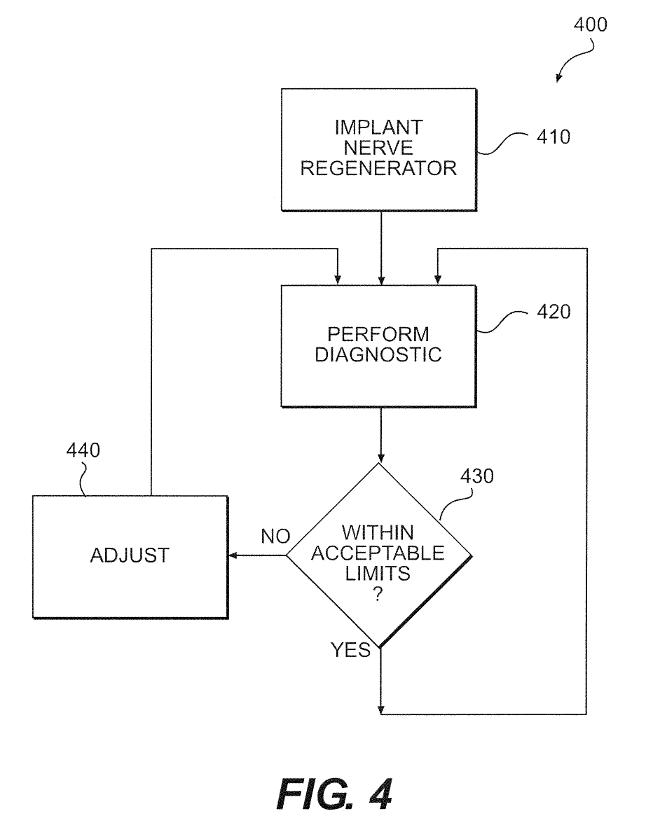

Consistent with certain aspects of the present disclosure, FIGS. 4-7 illustrate exemplary methods for promoting nerve regeneration in central and peripheral nervous systems of mammals. FIG. 4 provides a flowchart 400 depicting an exemplary method for performing nerve regeneration treatments based on diagnostic analysis of patient data associated with the administration of the nerve regeneration treatments. As illustrated, a nerve regeneration device, such as nerve regenerator 100″ shown in FIGS. 1A and 1B, may be implanted in the vicinity of damaged and/or severed nerves (Step 410). According to one embodiment, nerve regenerator 100″ may be fully implanted beneath the skin of the patient during a surgical procedure. Control module 101 may be located at or near the surface of the skin of the patient to allow easy access to fluid port 102. During the implantation procedure, leads 150, which may include one or more electrodes 160 and associated conductors, sensors 173, transducers 170, and/or fluid delivery tubes 108, may be strategically positioned at or near damaged nerves, and may be adapted to deliver multiple types of nerve regeneration treatments to the damaged nerves and/or the surrounding nerve tissue.

Once nerve regenerator 100″ has been implanted, nerve regeneration system 200 may initiate nerve regeneration treatments. Nerve regeneration treatments may include electrical stimulation, chemical treatment, stem cell delivery, and/or a combination of these treatments of the damaged nerve cells. These treatments may be administered in accordance with a “standard” nerve regeneration treatment regimen, which may include a default or general nerve regeneration treatment strategy. According to one exemplary embodiment, nerve treatment may include electromagnetic stimulation to promote nerve growth coupled with the administration of a chemical nerve growth agent and/or stem cells, which may enhance the effectiveness of the electromagnetic treatment.

Nerve regeneration system 200 may be configured to perform diagnostic tests to determine the effectiveness of the nerve regeneration treatments (Step 420). For example, during a nerve regeneration treatment session, a diagnostic tool, such as tools 600′ or 600″ of FIGS. 3A and 3B, respectively, may administer a series of diagnostic tests. Sensors 173 associated with nerve regenerator 100″ may measure electrical signals (nerve action potentials, EMG, ECoG, LFP, EEG and/or other neural signals) in response to the administration of the diagnostic test. After each measurement, a health care provider, doctor, or technician may adjust one or more parameters of the nerve regeneration treatment and repeat the diagnostic test. The test results may be compared with a previously performed test to determine the impact of the change on the neurological response to determine if additional adjustment to the treatment parameter is required. This process may be repeated to identify the nerve regeneration treatment settings that achieve the strongest response from one or more nerves or sets of nerves.

Alternatively or in addition to measuring a nerve responsiveness under various treatment settings, nerve regenerator 100″ may monitor a nerve response to internal stimulants, such as stimulations (e.g., light, vibrations, magnetic fields, small signal electrical signals, etc.) generated by transducers 170. According to one exemplary embodiment, one or more electrodes 160 may be configured to generate a small DC current, which may be dispersed through tissue to one or more different electrodes 160 in an area proximate damaged nerve tissue. One or more sensors 173 may be configured to measure one or more physical, chemical, physiologic, and/or electrical responses provided by the damaged nerves. The strength of the responses may be measured over time to identify treatment parameters that result in the most effective nerve growth response.

After the diagnostic data has been collected, the data may be compared with acceptable limits to determine if settings associated with the current nerve regeneration treatments are effective (Step 430). For example, cellular or multicellular electrical signals collected in response to a diagnostic stimulus may be compared with predetermined threshold response ranges or limits. If the collected data reveals an electrical signal level that is inconsistent with a predetermined threshold signal level (e.g., outside an acceptable range) (Step 430: No), one or more nerve regeneration treatment parameters may be adjusted (Step 440). The diagnostic process may then be repeated, either automatically (i.e., with nerve regeneration system 200 set to closed-loop or “automatic” mode) or manually (i.e., when nerve regeneration system 200 is set in manual mode).

If, on the other hand, the collected diagnostic data is consistent with predetermined threshold data (i.e., within acceptable ranges) (Step 430: Yes), the diagnostic process may be repeated (either periodically and/or continuously). As treatments progress, the patient's response to the treatments may change. Thus, certain treatment parameters that may be effective at earlier stages of nerve regeneration treatment may become less effective as treatment progresses. By responsively modifying threshold parameters based on diagnostic analysis of the effectiveness of nerve regeneration settings, nerve regeneration system 200 may optimize the treatment regimen to adapt to changes in a patient's response to the treatments.

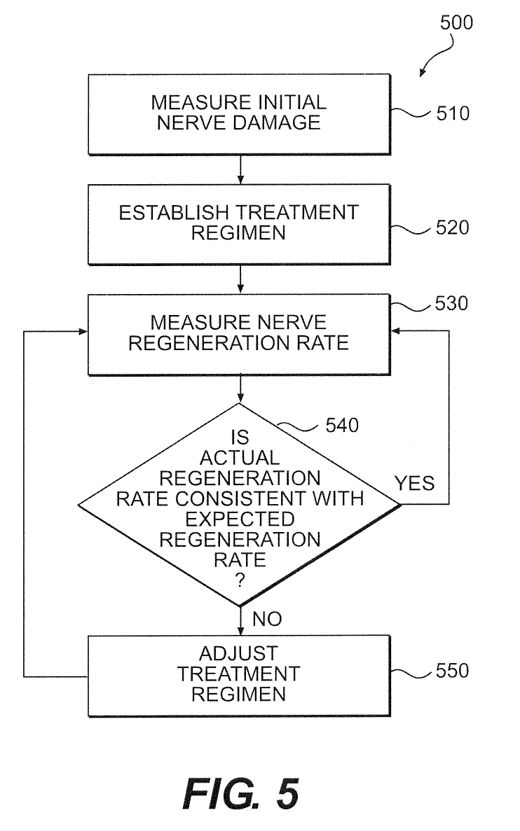

FIG. 5 provides a flowchart 500 depicting an exemplary nerve regeneration treatment optimization process consistent with the disclosed embodiments. The process commences by measuring initial damage to the nerves of a patient (Step 510). Initially, this measurement may be performed manually during, for example, a neurologist examination after a nerve-damaging injury is sustained. This measurement may be performed more precisely after implantation and/or installation of nerve regenerator 100″ near the damaged cells of the patient.

According to one embodiment, one or more sensory stimulants (e.g., pin-prick test, light touch test, electrical stimulation test, nerve conduction test, etc.) may be administered, and a neural response may be measured by sensors 173 associated with nerve regenerator 100″. The strength of the received neural response may be measured to determine the extent of the nerve damage. For example, an electrical signal generated by the damaged nerve(s) in response to the stimulation may be compared with predetermined threshold ranges, wherein each range may be indicative of a relative health of a damaged nerve. In another example, nerve responses to stimulation may result in a chemical, hormonal or other physiologic change in the surrounding tissue. This chemical response may be measured and compared with predetermined chemical response ranges to determine the extent of nerve damage. In either example provided above, those skilled in the art will recognize that a weak electrical or chemical response may be characteristic of severely damaged nerves, while stronger responses are typically indicative of healthy nerves.

Once the initial nerve damage has been assessed, a nerve regeneration treatment regimen may be established (Step 520). The treatment regimen may include, for example, providing electromagnetic stimulation to the damaged nerve tissue (e.g., in the form of UV, RF, microwave, and/or optical radiation, etc.), delivering nerve regeneration agents (e.g., vitamins, steroids, proteins, growth factor, etc.) to the damaged nerves, or a combination of chemical and electrical treatments. The treatment regimen may be established manually by a health care professional (e.g., neurologist, nurse, etc.).

Alternatively, nerve regenerator 100″ may include one or more prescribed treatment regimen programs stored in memory. As such, nerve regenerator 100″ may automatically select a prescribed treatment regimen based on the initial damage assessment. For instance, if the measured electrical voltage level received in response to a diagnostic stimulation of damaged nerves is within a range corresponding to 80-90% of lost sensory function, a prescribed treatment regimen corresponding to this level of loss of sensory function may be selected and administered by nerve regenerator 100″.

As nerve functionality is restored, nerves may begin to respond differently to treatments. Thus, while a particular type or frequency of electromagnetic stimulation may promote fast growth in early stages of nerve regeneration, a different type or frequency of stimulation may be more effective in promoting growth in later stages of nerve regeneration. Additionally, because different patients may respond differently to the same treatment regimen, it may be advantageous to periodically measure the effectiveness of the treatment regimen on the nerve regeneration rate. Accordingly, nerve regenerator 100″ may measure the nerve regeneration rate during the administration of the nerve regeneration treatment (Step 530).

The nerve regeneration rate may be measured using multiple techniques. According to one embodiment, when initially implanting nerve regenerator 100″ and/or electrodes 160 in the body of a patient, a physician may record the precise placement of each electrode 160 relative to the damaged nerve(s). During treatments, nerve regenerator 100″ may provide pulses of electromagnetic energy at a particular test frequency. Sensors 173 may detect the energy reflected from the test pulses and, based on the time it takes to receive the reflected energy, nerve regenerator 100″ may estimate a new position of the nerve(s). This position may be compared with the original position of the nerve to determine the amount of growth that the nerve has experienced.

According to another embodiment, nerve regenerator 100″ may deposit a fluorescent, luminescent, or photo-sensitive dying agent into damaged nerve tissue. Using an LED or other type of radiation transducer 170 provided on one or more leads 150, nerve regenerator 100″ may activate the dye and measure the reactive response. Alternatively or additionally, fluid delivery device 107 may dispense a radiolabeled isotope or other radiographic material that, when imaged by a fluoroscope, may aid in visualizing nerves and/or nerve growth (e.g. a radiolabeled substance which is absorbed by and/or attaches to a nerve axon). This response may be analyzed by interrogator 210 and compared with historical responses to determine the nerve regeneration rate.

The nerve regeneration rate may be compared with an expected regeneration rate to determine the effectiveness of a current treatment regimen in developing the damaged nerves (Step 540). The expected regeneration rate may be predetermined based on historical nerve growth rates in controlled tests. If the nerve regeneration rate is consistent with the expected rate (Step 540: Yes), the current treatment regimen may be retained and the system may continue to monitor the nerve regeneration rate to ensure the maintenance of a desired nerve regeneration schedule.

If, however, the nerve regeneration rate is not consistent with expectations (Step 540: No), one or more treatment parameters may be adjusted. For example, a health care professional may manually update the dosage of the therapeutic drugs and/or one or more parameters associated with the electromagnetic stimulating treatment. As an alternative or in addition to the manual updates provided by a health care professional, nerve regenerator 100″ may be configured to automatically adjust the treatment parameters. Once the treatment parameters have been adjusted, the system may implement the adjusted treatment regimen and continue measuring nerve regeneration rate. This process may be repeated to ensure that treatment parameters are maintained so as to affect optimal development of damaged nerves.

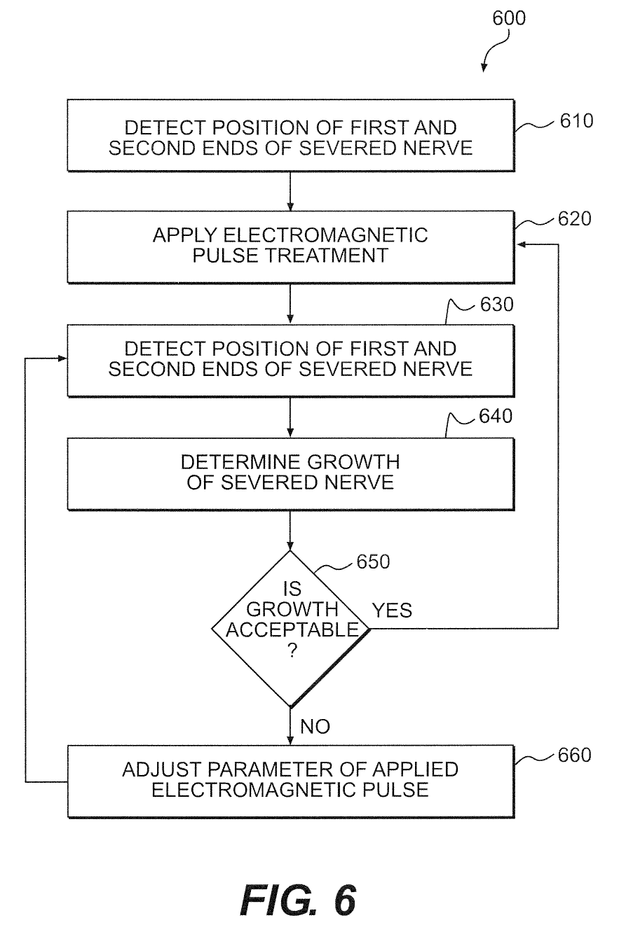

FIG. 6 provides a flowchart 600 depicting an exemplary method for repairing severed nerves in accordance with certain disclosed embodiments. This method may also be implemented to repair a damaged nerve by re-connecting or growing a damaged axon that is still connected to one nerve to an axon or neuron associated with a second nerve, regardless of whether that axon was originally connected to the second nerve. As illustrated in FIG. 6, positions of first and second ends of a severed nerve may be detected (Step 610). According to one embodiment, one or more electrodes 160 may each provide a diagnostic electromagnetic pulse to damaged nerve tissue. Each end of the severed nerve may generate an electrical signal in response to the electromagnetic pulse, which may be collected by one or more sensors 173 of nerve regenerator 100″. Because each sensor 173 may be located at a different distance from each end of the severed nerve, a position of each end of the severed nerve may be detected through analysis of the various detected signals received by sensors 173.

Once ends of the severed nerve have been located, nerve regenerator 100″ may provide electromagnetic nerve treatment to stimulate growth of the axons (Step 620). As explained, electromagnetic nerve treatment may include DC fields such as constant current DC fields, UV, RF, microwave, millimeter wave, optical, or any other type of electrical field or other electromagnetic radiation that may promote the growth of damaged nerves.

During electromagnetic nerve treatments, nerve regeneration system 200 may detect the position of first and second ends of the severed nerve (Step 630) and determine a growth of the severed nerve (Step 640). For example, during periods when electrodes are not administering nerve regeneration stimulation, they may provide electromagnetic pulses for locating the ends of the severed nerves. Nerve growth rate may be calculated by determining the change in position of the detected nerve ending position with a previously detected position of the nerve ending.

The amount of growth and/or the growth rate may be evaluated to determine whether the nerve regeneration treatment is producing acceptable results (Step 650). According to one embodiment, the growth rate may be compared with a predetermined nerve growth rate. If the growth rate is not acceptable (i.e., inconsistent with predetermined growth levels) (Step 650: No), nerve regenerator 100″ and/or a healthcare professional may adjust a parameter associated with the applied electromagnetic growth treatment (Step 660). As explained, this may include modifying a direction and/or strength of the applied electromagnetic field, a voltage or current level associated with the electromagnetic field, a frequency or duty cycle of the applied field, or any other parameter associated with the electromagnetic treatment.

If, on the other hand, the growth rate is acceptable (Step 650: Yes), the current treatment parameters may be retained. Accordingly, nerve regenerator 100″ may continue administering electromagnetic pulse treatment and monitoring a nerve regeneration rate corresponding to the applied therapeutic treatment.

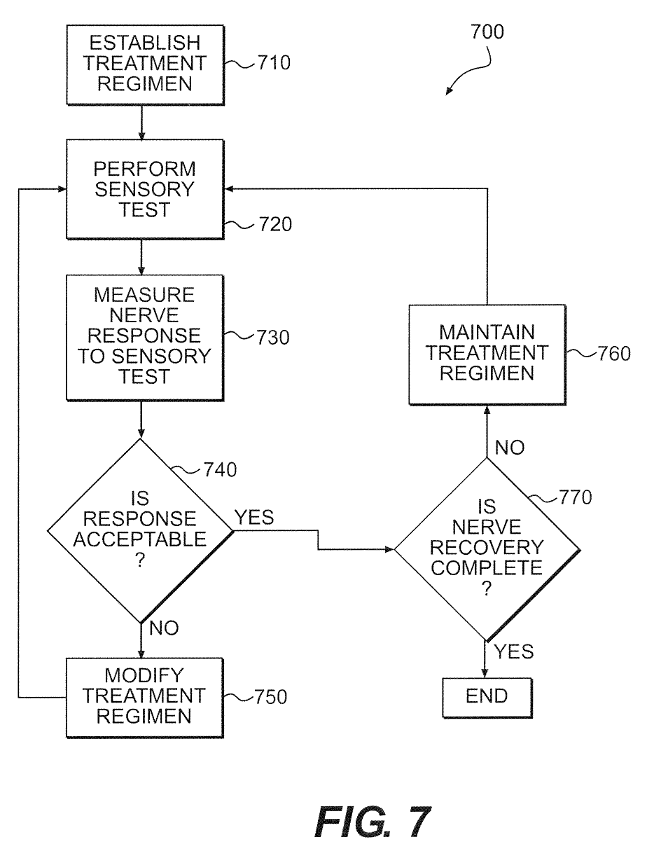

FIG. 7 provides a flowchart 700 depicting an exemplary method for integrating the diagnostic tools of FIGS. 3A and 3B into nerve regeneration system 200. According to one embodiment, interrogator 210 and/or control module 101 of nerve regenerator 100″ may be in wireless communication with control module 601′ or 601″ associated with diagnostic tools 600′ or 600″. As such, nerve regenerator 100″ and/or interrogator 210 may periodically monitor nerve response to an external sensory test to determine the effectiveness of a nerve regeneration treatment.

As illustrated in FIG. 7, the process may begin upon establishment of a nerve treatment regimen (Step 710). A health care practitioner or doctor may establish the initial treatment settings, based on historical treatment data gathered from treatments of previous patients, laboratory tests, and/or medical studies.

In order to optimize the effectiveness of the nerve regeneration treatment, nerve regenerator 100″ and/or interrogator 210 may periodically request sensory tests to determine if the current treatment parameters are effective in restoring the sensory functions of the patient. Accordingly, a sensory test command may be provided by nerve regenerator 100″ and/or interrogator 210 to one or more of diagnostic tools 600′ and 600″. In response to the command, the one or more diagnostic tools may administer the sensory test on the patient (Step 720). In response to the sensory test, diagnostic tools and/or sensors 173 of nerve regenerator 100″ may monitor the biological, physiological, chemical, and/or electrical neurological response of the patient to the test (Step 730). In addition to monitoring the internal and/or physical responses to the sensory test, a health care provider or lab technician may request feedback from the patient to determine whether the sensory test produced a physical sensation for the patient.

According to one embodiment, nerve regenerator 100″, interrogator 210, and/or the patient may determine if the response to the sensory test is acceptable (Step 740). If the response is acceptable (Step 740: Yes) and the nerve recovery process is complete (Step 760: Yes), the nerve regeneration process may be terminated. If, on the other hand, the response is acceptable (Step 740: Yes), but the damaged nerves have not yet fully recovered (Step 760: No), the treatment regimen may be continued (Step 770).

If the nerve response to the sensory test is not acceptable (Step 740: No), a health care provider may modify one or more treatment parameters (e.g., one or more parameters associated with drug delivery and/or electromagnetic stimulation) (Step 750) and repeat the diagnostic process (Steps 720-740).

Alternatively, if nerve regenerator 100″ is set to operate in an automated (i.e., closed-loop) mode, nerve regenerator 100″ may be programmed to adjust treatment parameters without requiring manual configuration by a health care provider. As such, health care professionals may only need to periodically monitor the nerve regeneration system 200 to ensure that the system is operating normally. When operating in an automated mode, nerve regenerator 100″ may be adapted to provide a periodic status update (e.g., “heartbeat” signal) to interrogator 210, which may notify a health care professional of problems associated with nerve regenerator 100″ and/or treatments administered thereby.

According to one embodiment, interrogator 210 may embody a wireless paging device, PDA, or cell phone and may be configured to receive status updates associated with treatments via wireless internet or a cellular network. In addition, certain treatment parameters may be adjusted remotely by interrogator 210 or other computer system using a network accessible wireless web-interface in data communication with nerve regenerator 100″.

In certain situations, interrogator 210 may be configured to perform a permission routine for preventing unauthorized users from accessing patient data and/or modifying treatment settings. The permission routine may employ one or more of: a password; a restricted user logon function; a user ID; an electronic key; a electromechanical key; a mechanical key; a specific Internet IP address; and other means of confirming the identify of one or more operators prior to allowing a secure operation to occur.

Although certain processes and features associated with the disclosed embodiments may be illustrated and discussed in relation to nerve regeneration treatments for damaged sensory nerves associated with peripheral nervous system, they may be applicable to regenerating nerve cells associated with the central nervous system. Accordingly, nerve regenerator 100″ and/or leads associated therewith, may be disposed at or near the brain and/or spinal column of a patient in order to effectively administer nerve regenerative treatments thereto.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth below not be construed as being order-specific unless such order specificity is expressly stated in the claim.

What is claimed is:

1. A nerve regeneration system comprising:
a lead configured to be placed in a body proximate a damaged nerve, a portion of the lead being configured to stimulate the damaged nerve; and
a control module configured to monitor a signal indicative of the nerve's response to the stimulation and adjust a parameter of the stimulation in response to the monitored signal.

2. The system of claim 1, wherein the stimulation comprises a therapeutic electric signal.

3. The system of claim 2, wherein the parameter of the stimulation comprises a parameter associated with the electric signal.

4. The system of claim 3, wherein the parameter comprises one or more of strength, direction, current, or voltage of the electric signal.

5. The system of claim 1, further comprising an electrode coupled to the lead and configured to deliver electric stimulation to the damaged nerve.

6. The system of claim 5, wherein the electrode comprises a plurality of electrodes and the parameter comprises one or more of a number, a sequence, or a combination of electrodes to be energized to deliver electric stimulation.

7. The system of claim 5, further comprising a conductor for connecting the electrode to the control module.

8. The system of claim 1, wherein the control module is enclosed in a substantially sealed housing and the lead extends from the housing.

9. The system of claim 8, wherein multiple leads extend from the housing.

10. The system of claim 8, wherein the control module is configured to communicate with an external device.

11. The system of claim 1, wherein the control module is surgically implanted within the body.

12. The system of claim 1, wherein the control module is partially implanted in the body such that at least a portion of the control module is accessible from outside the body.

13. The system of claim 1, wherein the control module comprises a fluid delivery device configured to provide a therapeutic fluid to the damaged nerve.

14. The system of claim 13, wherein the control module comprises a fluid port for supplying fluid to the fluid delivery device.

15. The system of claim 13, wherein the lead is configured to deliver an electrical signal, and the system further comprises a second lead configured to deliver the therapeutic fluid to the damaged nerve.

16. The system of claim 13, wherein the stimulation is delivered by the therapeutic fluid.

17. The system of claim 16, wherein the parameter comprises a delivery parameter associated with the delivery of therapeutic fluid.

18. The system of claim 17, wherein the delivery parameter comprises one or more of a schedule, rate, or dosage of a therapeutic fluid.

19. The system of claim 13, wherein the lead comprises a tube in fluid communication with the fluid delivery device and configured to deliver the therapeutic fluid to the damaged nerve.

20. The system of claim 13, wherein the control module comprises a reservoir in fluid communication with the fluid delivery device for storing a supply of the therapeutic fluid.

21. The system of claim 13, wherein the therapeutic fluid comprises at least one of a nerve growth agent, an anti-infection agent, and a pain reducing agent.

22. The system of claim 1, wherein the lead comprises an expandable member configured to secure the lead proximate the damaged nerve.

23. The system of claim 22, wherein the expandable member comprises an inflatable balloon.

24. The system of claim 23, wherein the control module is configured to control the inflation of the balloon.

25. The system of claim 22, further comprising a fluid delivery device adapted to inflate the inflatable balloon.

26. The system of claim 1, further comprising a sensor coupled to the lead and configured to detect the signal indicative of the nerve's response to the stimulation.

27. The system of claim 26, wherein the sensor comprises one or more of: a physiologic sensor, a cellular sensor, an EEG sensor, an EKG sensor, an EMG sensor, a blood sensor, a glucose sensor, a temperature sensor, a radiation sensor, a magnetic sensor, and a chemical sensor.

28. The system of claim 26, wherein the damaged nerve is a severed nerve having a first end and a second end, and the sensor comprises a first position sensor and a second position sensor, the first position sensor adapted to detect a position of the first end and the second position sensor adapted to detect a position of the second end.

29. The system of claim 28, wherein:
the control module delivers an electromagnetic signal proximate the severed nerve; and
the sensor monitors a reflection of the electromagnetic signal.

30. The system of claim 28, wherein the control module is configured to:
analyze the detected positions of the first and second ends to determine a regeneration rate of the severed nerve; and
modify the parameter based on the regeneration rate of the severed nerve.

31. The system of claim 26, further comprising a transducer coupled to the lead and adapted to deposit a tagging agent proximate the damaged nerve.

32. The system of claim 31, wherein the sensor is configured to detect a growth of the damaged nerve indicative of a change in a position of the tagging agent.

33. The system of claim 31, wherein the sensor is configured to detect a reaction of the tagging agent to an electromagnetic stimulus.

34. The system of claim 31, wherein the tagging agent comprises a fluorescent or luminescent material.

35. The system of claim 31, wherein the tagging agent comprises an RFID device.

36. A nerve regeneration system comprising:
a nerve regeneration module comprising at least one lead implanted in a body proximate a damaged nerve, the nerve regeneration module being configured to:
administer a nerve regeneration treatment to the damaged nerve; and
detect a patient response to the nerve regeneration treatment; and
an interrogator communicatively coupled to the nerve regeneration module and configured to modify a parameter of the nerve regeneration treatment based on the detected patient response.

37. The system of claim 36, wherein the nerve regeneration module is fully implanted in the body of a patient while the interrogator is outside the body.

38. The system of claim 37, wherein the interrogator is configured to modify the parameter of the nerve regeneration treatment after implantation of the nerve regeneration module.

39. The system of claim 36, wherein the interrogator is wirelessly coupled to the nerve regeneration module.

40. The system of claim 36, wherein the interrogator comprises a wireless communication device.

41. The system of claim 40, wherein the interrogator comprises a personal data assistant (PDA) or a wireless telephone.

42. The system of claim 36, wherein the nerve regeneration module is partially implanted in the body of a patient.

43. The system of claim 36, wherein the lead comprises an expandable member configured to secure the lead proximate the damaged nerve.

44. The system of claim 43, wherein the expandable member comprises an inflatable balloon.

45. The system of claim 36, further comprising a protective sheath surrounding the at least one lead.

46. The system of claim 36, further comprising a barb for securing the at least one lead within the body of the patient.

47. The system of claim 46, wherein the barb is retractable.

48. The system of claim 36, wherein the nerve regeneration module comprises a power supply configured to generate an electromagnetic signal for stimulating the damaged nerve.

49. The system of claim 48, wherein the at least one lead comprises one or more electrodes electrically coupled to the power supply, the electrode being configured to deliver the electromagnetic signal to the damaged nerve.

50. The system of claim 49, wherein the one or more of the electrodes are disposed along a length of the at least one lead.

51. The system of claim 36, wherein the nerve regeneration module comprises a fluid delivery system configured to deliver fluid proximate the damaged nerve.

52. The system of claim 51, wherein the fluid delivery system comprises a reservoir for storing fluid associated with the fluid delivery system.

53. The system of claim 51, wherein the fluid comprises a therapeutic fluid and the fluid delivery system is configured to deliver the therapeutic fluid via a fluid delivery tube.

54. The system of claim 53, wherein the fluid delivery tube is routed within the at least one lead.

55. The system of claim 53, wherein the therapeutic fluid comprises a nerve growth agent.

56. The system of claim 51, wherein the fluid delivery system is configured to supply inflating fluid to an expandable member coupled to the at least one lead.

57. The system of claim 51, wherein the fluid delivery system may comprise a syringe associated with the at least one lead.

58. The system of claim 36, wherein the nerve regeneration module comprises a sensor configured to monitor at least one of a biological, physiological, chemical, and electrical conditions associated with the damaged nerve.

59. The system of claim 58, wherein the sensor is coupled to the at least one lead.

60. The system of claim 59, wherein the sensor comprises a chemical sensor adapted to collect data indicative of a hormone level.

61. The system of claim 59, wherein the sensor comprises an electrical signal detector adapted to detect neurological signals associated with the damaged nerve.

62. The system of claim 59, wherein the sensor comprises a heart rate monitor.

63. The system of claim 59, wherein the sensor comprises a temperature sensor.

64. The system of claim 36, further comprising a transducer communicatively coupled to the nerve regeneration module and configured to administer a diagnostic test when prompted by at least one of the nerve regeneration module and the interrogator.

65. The system of claim 64, wherein the transducer comprises an external diagnostic tool adapted to administer a sensory test to a portion of the body.

66. The system of claim 65, wherein the sensory test comprises a pin-prick test.

67. The system of claim 65, wherein the sensory test comprises a light-touch test.

68. The system of claim 64, wherein the transducer is configured to deploy a tagging agent proximate damaged nerves.

69. The system of claim 68, wherein the tagging agent comprises a frequency-responsive dying agent.

70. The system of claim 69, wherein the transducer is further configured to deliver an electrical signal at an appropriate frequency to activate the frequency-responsive dying agent.

71. The system of claim 36, wherein the nerve regeneration treatment comprises transmitting an electromagnetic signal to the damaged nerve to stimulate the damaged nerve.

72. The system of claim 71, wherein the parameter comprises at least one of a power level, a frequency, a field strength, and field direction associated with the electromagnetic signal.

73. The system of claim 36, wherein the nerve regeneration treatment comprises delivering a therapeutic fluid proximate the damaged nerve.

74. The system of claim 73, wherein the parameter comprises at least one of a dosage and a schedule associated with the therapeutic fluid delivery.

75. A method for regenerating a damaged nerve comprising:
providing a therapeutic stimulation to a damaged nerve;
monitoring a signal indicative of the nerve's response to the stimulation; and
adjusting a stimulation parameter in response to the monitored signal.

76. The method of claim 75, wherein the providing, the monitoring, and the adjusting are performed by an integrated device.

77. The method of claim 75, wherein providing the therapeutic stimulation comprises:
implanting a stimulation device within a body; and
adjusting the stimulation parameter after implantation.

78. The method of claim 77, wherein the stimulation device comprises a control module having a substantially sealed housing with a lead extending from the housing.

79. The method of claim 78, further comprising fixing the lead in the body of a patient proximate to the damaged nerve.

80. The method of claim 78, further comprising providing the signal to an external diagnostic device.

81. The method of claim 78, wherein adjusting the stimulation parameter comprises automatically adjusting the stimulation parameter based on the monitored signal.

82. The method of claim 78, wherein adjusting the stimulation parameter comprises manually adjusting the stimulation parameter based on the monitored signal.

83. The method of claim 78, wherein adjusting the stimulation parameter comprises:
comparing the monitored signal with a predetermined threshold value;
displaying results of the comparison on a display of the external device; and
receiving a user command for adjusting the stimulation parameter.

84. The method of claim 78, wherein adjusting the stimulation parameter comprises:
comparing the monitored signal with a predetermined threshold value; and
performing a predetermined adjustment routine if the monitored signal exceeds an acceptable deviation limit from the predetermined threshold value.

85. The method of claim 75, wherein providing a therapeutic stimulation comprises delivering an electromagnetic signal to the damaged nerve.

86. The method of claim 85, wherein adjusting a stimulation parameter comprises adjusting at least one of a power level, a frequency, a field strength, and a field direction associated with the electromagnetic signal.

87. The method of claim 75, wherein providing a therapeutic stimulation comprises delivering a therapeutic fluid to the damaged nerve.

88. The method of claim 87, wherein adjusting a stimulation parameter comprises adjusting a dosage of the therapeutic fluid.

89. The method of claim 88, wherein adjusting a stimulation parameter comprises adjusting a schedule for administering the therapeutic fluid.

90. The method of claim 85, wherein the therapeutic fluid comprises a nerve growth agent.

91. The method of claim 75, wherein providing a therapeutic stimulation comprises providing a combination of electromagnetic stimulation and chemical nerve treatment therapy to the damaged nerve.

92. The method of claim 75, wherein providing a therapeutic stimulation comprises providing a combination of electromagnetic stimulation and stem cell nerve treatment therapy to the damaged nerve.

93. The method of claim 75, wherein monitoring a signal comprises monitoring an electric signal emitted by the damaged nerve in response to the therapeutic stimulation.

94. The method of claim 75, wherein monitoring a signal comprises monitoring a hormone level of nerve tissue surrounding the damaged nerve.

95. The method of claim 75, wherein monitoring a signal comprises:
providing an electrical test signal to the damaged nerve;
measuring the nerve's response to the test signal; and
determining a current location of a portion of the damaged nerve based on the measured response to the test signal.

96. The method of claim 95, wherein monitoring a signal comprises calculating a growth of the nerve as the difference between the current location of the portion of the damaged nerve and a previous location of the portion of the damaged nerve.

97. A method for promoting nerve regeneration comprising:
administering a nerve regeneration treatment to a damaged nerve;
monitoring a growth associated with the damaged nerve;
comparing the monitored growth with a predetermined value; and
determining whether to adjust a nerve regeneration treatment based on the comparison of the monitored growth with the predetermined value.

98. The method of claim 97, wherein determining whether to adjust the nerve regeneration treatment comprises:
modifying a nerve regeneration treatment parameter if the monitored growth is less than the predetermined value; and
maintaining the nerve regenerating treatment if the monitored growth is greater than the predetermined value.

99. The method of claim 98, wherein administering the nerve regeneration treatment comprises providing an electromagnetic signal for electrically stimulating the damaged nerve.

100. The method of claim 99, wherein modifying the nerve regeneration treatment parameter comprises adjusting at least one of a power level, frequency, field strength, and field direction associated with the electromagnetic signal.

101. The method of claim 98, wherein administering the nerve regeneration treatment comprises delivering a therapeutic fluid to the damaged nerve.

102. The method of claim 101, wherein modifying a nerve regeneration treatment parameter comprises adjusting a dosage associated with the therapeutic fluid.

103. The method of claim 101, wherein modifying a nerve regeneration treatment parameter comprises adjusting a schedule for administering the therapeutic fluid.

104. The method of claim 97, wherein monitoring the growth associated with one or more damaged nerves comprises:
delivering an electromagnetic test pulse to the damaged nerve;
detecting a neurological response from the damaged nerve in response to the test pulse; and
estimating the growth based on the detected neurological response from the damaged nerve.

105. The method of claim 104, wherein estimating the growth comprises comparing the detected neurological response data with previously detected neurological response data.

106. The method of claim 97, wherein monitoring the growth comprises:
providing a first electromagnetic test signal from a first location relative to the damaged nerve;
providing a second electromagnetic test signal from a second location relative to the damaged nerve;
determining a position of an end of a damaged nerve based on the difference between the first electromagnetic test signal and the second electromagnetic test signal; and
comparing the position of the end of the damaged nerve with a previous position of the end of the damaged nerve to determine the growth of the damaged nerve.

107. A nerve regeneration system comprising:
at least one lead implanted in a body proximate a damaged nerve, and
a control module connected to the at least one lead, wherein the control module is configured to:
administer a nerve regeneration treatment to the damaged nerve through the lead;
monitor the growth associated with the damaged nerve; and
determine whether to adjust a nerve regeneration treatment parameter based on the comparison of the monitored growth with the predetermined value.

108. The system of claim 107, wherein the nerve regeneration treatment comprises a therapeutic electric signal.

109. The system of claim 108, wherein the nerve regeneration treatment parameter comprises a parameter associated with the electric signal.

110. The system of claim 109, wherein the parameter comprises one or more of strength, direction, current, and voltage of the electric signal.

111. The system of claim 107, further comprising an electrode coupled to the lead and configured to deliver electric nerve regeneration treatment to the damaged nerve.

112. The system of claim 111, wherein the electrode comprises a plurality of electrodes and the parameter comprises one or more of a number, a sequence, and a combination of electrodes to be energized to deliver electric nerve regeneration treatment.

113. The system of claim 111, further comprising a conductor for connecting the electrode to the control module.

114. The system of claim 107, wherein the control module is enclosed in a substantially sealed housing and the lead extends from the housing.

115. The system of claim 114, wherein multiple leads extend from the control module.

116. The system of claim 114, wherein the control module is configured to communicate with an external device.

117. The system of claim 107, wherein the control module is surgically implanted within the body.

118. The system of claim 107, wherein the control module is partially implanted in the body such that at least a portion of the control module is accessible from outside the body.

119. The system of claim 107, wherein the control module comprises a fluid delivery device configured to provide a therapeutic fluid to the damaged nerve.

120. The system of claim 119, wherein the control module comprises a fluid port for supplying fluid to the fluid delivery device.

121. The system of claim 119, wherein the lead is configured to deliver an electrical signal, and the system further comprises a second lead configured to deliver the therapeutic fluid to the damaged nerve.

122. The system of claim 119, wherein the stimulation is delivered by the therapeutic fluid.

123. The system of claim 122, wherein the parameter comprises a delivery parameter associated with the delivery of therapeutic fluid.

124. The system of claim 123, wherein the delivery parameter comprises one or more of a schedule, rate, or dosage of a therapeutic fluid.

125. The system of claim 119, wherein the lead comprises a tube in fluid communication with the fluid delivery device and configured deliver the therapeutic fluid to the damaged nerve.

126. The system of claim 119, wherein the control module comprises a reservoir in fluid communication with the fluid delivery device for storing a supply of the therapeutic fluid.

127. The system of claim 119, wherein the therapeutic fluid comprises at least one of a nerve growth agent, an anti-infection agent, and a pain reducing agent.

128. The system of claim 107, wherein the lead comprises an expandable member configured to secure the lead proximate the damaged nerve.

129. The system of claim 128, wherein the expandable member comprises an inflatable balloon.

130. The system of claim 129, wherein the control module is configured to control the inflation of the balloon.

131. The system of claim 128, further comprising a fluid delivery device adapted to inflate the inflatable balloon.

132. The system of claim 107, further comprising a sensor coupled to the lead and configured to detect the signal indicative of the nerve's response to the stimulation.

133. The system of claim 132, wherein the sensor comprises one or more of: a physiologic sensor, a cellular sensor, an EEG sensor, an EKG sensor, an EMG sensor, a blood sensor, a glucose sensor, a temperature sensor, a radiation sensor, a magnetic sensor, and a chemical sensor.

134. The system of claim 132, wherein the damaged nerve is a severed nerve having a first end and a second end, and the sensor comprises a first position sensor and a second position sensor, the first position sensor adapted to detect a position of the first end and the second position sensor adapted to detect a position of the second end.

135. The system of claim 134, wherein:
the control module delivers an electromagnetic signal proximate the severed nerve; and
the sensor monitors a reflection of the electromagnetic signal.

136. The system of claim 134, wherein the control module is configured to:
analyze the detected positions of the first and second ends to determine a regeneration rate of the severed nerve; and
modify the parameter based on the regeneration rate of the severed nerve.

137. The system of claim 132, further comprising a transducer coupled to the lead and adapted to deposit a tagging agent proximate the damaged nerve.

138. The system of claim 137, wherein the sensor is configured to detect a growth of the damaged nerve indicative of a change in a position of the tagging agent.

139. The system of claim 137, wherein the sensor is configured to detect a reaction of the tagging agent to an electromagnetic stimulus.

140. The system of claim 137, wherein the tagging agent comprises a fluorescent or luminescent material.

141. The system of claim 137, wherein the tagging agent comprises an RFID device.

142. A device for administering a neurological test to a portion of a patient's body comprising:
a probe;
a drive assembly configured to move the probe relative to a patient's body; and
a controller configured control the operation of the drive assembly to bring at least a portion of the probe in contact with the patient's body and to provide physical stimulation to the portion of the patient's body in accordance with a predetermined test parameter.

143. The device of claim 142, wherein the probe comprises a pin for pricking the skin of the patient.

144. The device of claim 143, wherein the drive assembly comprises a linear drive device for extending and retracting the pin from the skin of the patient.

145. The device of claim 142, wherein the controller is communicatively coupled to an external diagnostic device and configured to control the operation of the drive assembly in response to command signals from the external diagnostic device.

146. The device of claim 142, wherein the probe includes a rotatable member configured to rub the skin of the patient.

147. The device of claim 142, wherein the predetermined test parameter includes at least one of: a force applied by the drive assembly, an amount of pressure applied by the probe to the patient's body, an amount of movement of the probe relative to the patient's body, a duration of operation of the drive assembly, and a range of motion associated with the drive assembly.

148. The device of claim 142, further comprising a fixing member configured to fix the device relative to the patient's body.

149. The device of claim 148, wherein the fixing member comprises a band configured to wrap around a portion of the patient's body.

150. A method for administering a neurological test to a patient's body comprising:
establishing at least one test parameter for administering a neurological test to the skin of the patient;
stimulating a surface of the patient's skin according to the at least one test parameter; and
monitoring the patient's response to the stimulation.

151. The device of claim 150, wherein the at least one test parameter includes at least one of: a force applied by the drive assembly, an amount of pressure applied by the probe to the patient's body, an amount of movement of the probe relative to the patient's body, a duration of operation of the drive assembly, and a range of motion associated with the drive assembly.
